(19) United States
(12) Patent Application Publication
(10) Pub. No.: US 2025/0302341 A1
(43) Pub. Date: Oct. 2, 2025

(54) CONTINUOUS ANALYTE MONITORING DEVICE

(71) Applicants: Jiangsu Yuwell POCTech Biotechnology Co., Ltd., Zhenjiang (CN); JIANGSU YUEKAI BIOTECHNOLOGY CO., LTD., Nanjing (CN); Zhejiang POCTech Co., Ltd., Huzhou (CN)

(72) Inventors: Liang WANG, Zhenjiang (CN); Yanan ZHANG, Zhenjiang (CN)

(21) Appl. No.: 19/190,833

(22) Filed: Apr. 28, 2025

Related U.S. Application Data
(63) Continuation of application No. PCT/CN2024/122180, filed on Sep. 29, 2024.

(30) Foreign Application Priority Data
Mar. 29, 2024 (CN) 202410377162.0

(51) Int. Cl. A61B 5/145; A61B 17/34

(52) U.S. Cl. CPC A61B 5/14503; A61B 17/3468; A61B 2560/063

(57) ABSTRACT

A continuous analyte monitoring device includes an outer housing, a bottom housing, a monitoring assembly, and a sealing assembly. The outer housing has a first end and a second end opposite to the first end in a first direction. The second end has an engagement opening. The bottom housing is connected to the second end and has an implantation opening. The monitoring assembly includes a first monitoring unit inside the outer housing and a second monitoring unit at the bottom housing. The sealing assembly is configured to abut with the outer housing and/or the bottom housing and is located between the two electronic units to seal the engagement opening. The sealing assembly is configured to move along a second direction to unseal the engagement opening. The second direction is perpendicular to the first direction.

This application is a continuation of International Patent Application No. PCT/CN2024/122180, filed on Sep. 29, 2024, which claims priority to Chinese patent application No. 202410377162.0, titled “IN VIVO GLUCOSE MONITORING DEVICE” and filed with China National Intellectual Property Administration on Mar. 29, 2024, the entire contents of which are incorporated herein by reference.

### FIELD

The present disclosure relates to the field of medical device technologies, and more particularly, to a continuous analyte monitoring device.

### BACKGROUND

Continuous Glucose Monitoring (CGM) system is a medical device used to continuously monitor glucose levels of diabetic patients. Compared with conventional glucose monitoring methods, the CGM system provides continuous and detailed data about glucose levels, helping users better manage their glucose states.

When using a CGM product, the users need to place a housing of the CGM product on the skin and then press a trigger button. In this case, a puncture needle and a sensor pin inside the housing move towards and pierce the skin. An electrochemical reaction occurs between biological enzymes on the sensor and an interstitial fluid under the skin, and is converted into an electrical signal. The electrical signal is converted into glucose readings for the users. Electronic components for monitoring glucose levels in a host and transmitting signals to a display device are generally integrated within a monitoring assembly. After being implanted, the monitoring assembly adheres to a surface of the skin of the host to facilitate continuous monitoring.

A structure of the monitoring assembly typically includes two types, that is, integrated and split. An integrated monitoring assembly is assembled before leaving the factory, with a sensor and a signal transmitter inside the integrated monitoring assembly already connected to each other, in such a manner that the users do not need to assemble the integrated monitoring assembly and can use the integrated monitoring assembly directly. However, production costs of such an integrated monitoring assembly are relatively high. In addition, a sterilization process is likely to cause a sensor failure, which leads to a low yield and unguaranteed reliability.

A split monitoring assembly, although offering relatively high reliability, is not fully assembled at the factory and needs to be assembled by the users before use. Specifically, the monitoring assembly of this type is usually divided into two parts, one part is provided with the sensor, while the other is provided with the signal transmitter. These two parts are fixed in two housings, respectively. After receiving such a product, the users need to remove sealing films from the two housings, and then assemble the two housings together before proceeding with an implantation operation. This makes use steps of the product more cumbersome and increases learning costs of the users, resulting in unsatisfactory use experience.

### SUMMARY

The present disclosure provides a continuous analyte monitoring device to solve problems of cumbersome operation steps, inconvenient use, and susceptibility of internal assemblies to environmental contamination.

The present disclosure adopts the following technical solutions.

A continuous analyte monitoring device includes an outer housing having a first end and a second end opposite to the first end in a first direction, the second end having an engagement opening; a bottom housing connected to the second end and having an implantation opening; a monitoring assembly including a first monitoring unit fixed inside the outer housing and a second monitoring unit fixed at the bottom housing, the first monitoring unit including a sensor, and the second monitoring unit including a signal processing module; and a sealing assembly configured to abut with the outer housing and/or the bottom housing and located between the first monitoring unit and the second monitoring unit to seal the engagement opening, the sealing assembly being configured to move along a second direction to unseal the engagement opening and to allow the engagement opening to communicate with the implantation opening, the second direction being perpendicular to the first direction.

Preferably, a mounting channel is defined between the outer housing and the bottom housing, the mounting channel being configured to at least partially receive the sealing assembly and having a mounting opening that extends through the outer housing and/or the bottom housing along the second direction.

Preferably, the sealing assembly includes a sealing portion and a gripping portion extending from the sealing portion and protruding out of the mounting opening.

Preferably, the sealing assembly includes a connecting element and a sealing element fixed at the connecting element, the sealing element being configured to abut with the outer housing to seal the engagement opening.

Preferably, the outer housing and the bottom housing are configured to be rotatable relative to each other to switch between a first relative position in which the sealing assembly seals the engagement opening and a second relative position in which the sealing assembly unseals the engagement opening.

Preferably, in the first relative position, a first gap is formed between the outer housing and the bottom housing, and in the second relative position, a second gap is formed between the outer housing and the bottom housing, the first gap being smaller than the second gap.

Preferably, the sealing assembly includes a sealing portion sandwiched between the outer housing and the bottom housing and having a thickness greater than or equal to the first gap.

Preferably, the outer housing is provided with a fixing protrusion, and the bottom housing has a fixing groove having a locking position and an unlocking position spaced apart from the locking position circumferentially, a height difference existing between the locking position and the unlocking position in the first direction.

Preferably, the fixing groove includes an extension segment arranged between the locking position and the unlocking position and having a guiding surface smoothly connecting the locking position and the unlocking position.

Preferably, the first end is equipped with a trigger unit, and the outer housing is further equipped with a locking member inside the outer housing, the locking member being configured to abut between the trigger unit and the sealing assembly to prevent the trigger unit from moving towards the sealing assembly.

Preferably, the locking member extends in the first direction to form an acting end configured to abut with the sealing assembly and a stop end configured to lock the trigger unit.

Preferably, the outer housing is further equipped with a pushing member configured to apply a thrust in the first direction to the locking member.

Preferably, the continuous analyte monitoring device further includes a driving unit and a puncture unit that are disposed inside the outer housing. The first end is equipped with a trigger unit. Activating the trigger unit causes the driving unit to drive the puncture unit to move along the first direction, to cause the first monitoring unit to electrically connect the second monitoring unit and cause the sensor to be partially inserted into a host.

Preferably, the continuous analyte monitoring device further includes a driving unit and a puncture unit that are disposed inside the outer housing, the driving unit including at least two clamping portions, each of the at least two clamping portions being located at an outer side of the puncture unit to restrict a movement of the puncture unit relative to the clamping portion. The outer housing is further provided with a limiting sleeve inside the outer housing and having a limiting channel extending in the first direction. Each of the at least two clamping portions is located in the limiting channel and movable within the limiting channel in the first direction. When each of the at least two clamping portions moves relative to the limiting channel to a release position, the clamping portion releases the puncture unit to enable the puncture unit to move relative to the clamping portion in a third direction, the third direction being opposite to the first direction.

Preferably, the limiting sleeve includes a clamping segment and a release segment, in the first direction, a cross-sectional area of the clamping segment remaining constant and a cross-sectional area of the release segment gradually increasing, and each of the at least two clamping portions includes an inclined segment and a fixed segment, in the first direction, a cross-sectional area of the fixed segment remaining constant and a cross-sectional area of the inclined segment gradually increasing.

By adopting the above technical solutions, the present disclosure provides the following advantageous effects.

In the present disclosure, the driving unit and the first monitoring unit are fixed within the outer housing, while the second monitoring unit is fixed within the bottom housing. In this way, the continuous analyte monitoring device is divided into two modules. Components having relatively high sterilization requirements, such as the puncture unit and the sensor, are concentrated in one module (within the outer housing). Therefore, before leaving the factory, two modules, the outer housing and the bottom housing, can be separately sterilized using different processes or at different levels. After the sterilization, the outer housing and the bottom housing are assembled into an entirety, and the sealing assembly is arranged between the first monitoring unit and the second monitoring unit to isolate internal chambers of the outer housing and the bottom housing. This prevents an issue of a failure of a sensor or an electronic component during the sterilization. Moreover, since the bottom housing and the outer housing have been fixed in the factory, the user does not need to perform any fixing operations on the bottom housing and the outer housing before use, which reduces operation steps, improving the use experience.

Additionally, in the factory, the bottom housing and the outer housing have already been connected, with a space reserved between the bottom housing and the outer housing for a removal of the sealing assembly. In this way, when using the device, the user can realize communication between an inside of the outer housing and an inside of the bottom housing simply by moving the sealing assembly in the second direction, without assembling the bottom housing and the outer housing and without removing sealing films from the bottom housing and the outer housing separately. Therefore, use steps of the product are greatly simplified, which lowers operational difficulty, and makes preparation steps before use simpler and more convenient for the user. Further, before and after a movement of the sealing assembly, the outer housing and the bottom housing remain coaxially arranged, with only a gap left between the outer housing and the bottom housing. Consequently, when the sealing assembly is laterally removed, the bottom housing still shields the engagement opening of the in outer housing. Although the engagement opening is in communication with an ambient environment, a path for dust, bacteria, etc., to enter the engagement opening becomes more circuitous due to shielding of the bottom housing, which reduces a risk of bacterial entry into the outer housing from the engagement opening, enhancing cleanliness of assemblies inside the outer housing.

Since the sealing assembly moves in the second direction perpendicular to the first direction (an implantation direction), a movement of the sealing assembly does not affect a movement of the driving unit inside the outer housing. Therefore, a risk of interference between the sealing assembly and an implantation movement of the driving unit is lowered, which ensures that the driving unit can reliably perform the implantation movement after the sealing assembly unseals the engagement opening.

### BRIEF DESCRIPTION OF THE DRAWINGS

The accompanying drawings described here are used to provide a further understanding of the present disclosure and constitute a part of the present disclosure. Exemplary embodiments of the present disclosure and description thereof are used to explain the present disclosure, and do not constitute an improper limitation of the present disclosure.

FIG. 1 is a sectional view of a continuous analyte monitoring device according to an embodiment of the present disclosure.

FIG. 2 is a sectional view of a continuous analyte monitoring device with a sealing assembly removed in a second direction according to an embodiment of the present disclosure.

FIG. 3 is a sectional view of the continuous analyte monitoring device in FIG. 2, in which a driving unit is moved to a release position in a first direction.

FIG. 4 is a sectional view of the continuous analyte monitoring device in FIG. 3, in which a puncture unit is moved to a needle withdrawal position in a third direction.

FIG. 5 is a schematic structural view of a sealing assembly according to an embodiment of the present disclosure.

FIG. 6 is a schematic structural view of the sealing assembly in FIG. 5, viewed from another side.

FIG. 7 is a sectional view of a partial region of a continuous analyte monitoring device according to an embodiment of the present disclosure.

FIG. 8 is a sectional view of a partial region of a continuous analyte monitoring device according to another embodiment of the present disclosure.

FIG. 9 is a sectional view of a partial region of a continuous analyte monitoring device with a sealing assembly in FIG. 8 removed in a second direction.

FIG. 10 is a schematic structural view of a continuous analyte monitoring device according to an embodiment of the present disclosure.

FIG. 11 is a schematic structural view of a partial region of a continuous analyte monitoring device according to an embodiment of the present disclosure.

FIG. 12 is a schematic structural view of a driving unit according to an embodiment of the present disclosure.

1 outer housing; 11 engagement opening; 12 propelling member; 13 restoring member; 14 mounting channel; 141 mounting opening; 15 elastic claw; 16 engagement rib; 17 receiving groove; 18 fixing protrusion; 2 bottom housing; 21 implantation opening; 22 fixing groove; 221 locking position; 222 unlocking position; 223 extension segment; 23 guiding channel; 3 sealing assembly; 31 sealing portion; 32 gripping portion; 33 sealing element; 34 connecting element; 341 snapping hook; 35 shielding portion; 4 monitoring assembly; 41 first monitoring unit; 42 second monitoring unit; 5 driving unit; 51 clamping portion; 511 fixed segment; 512 inclined segment; 52 elastic rib; 6 puncture unit; 7 locking member; 71 stop protrusion; 72 outer flanging; 73 receiving space; 74 pushing member; 8 limiting sleeve; 81 limiting channel; 811 clamping segment; 812 release segment; 82 sliding groove; 9 trigger unit; 91 trigger button; 92 trigger rib.

### DETAILED DESCRIPTION

In order to more clearly explain the overall concept of the present disclosure, a detailed description is made below by way of example in conjunction with the accompanying drawings of the specification.

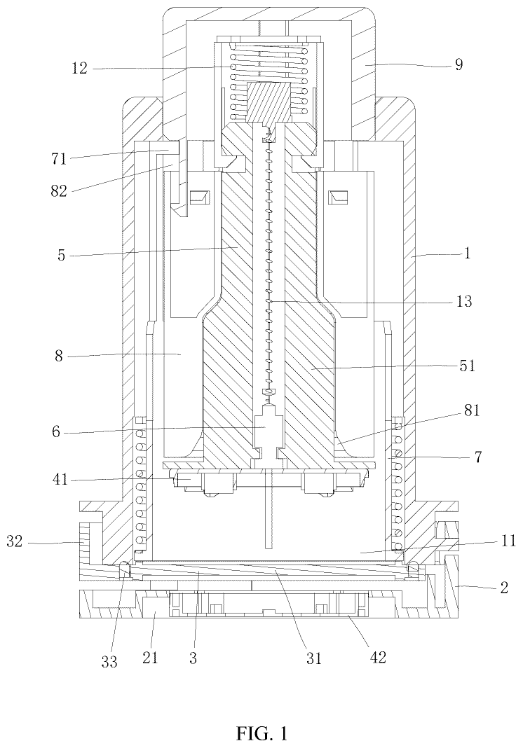

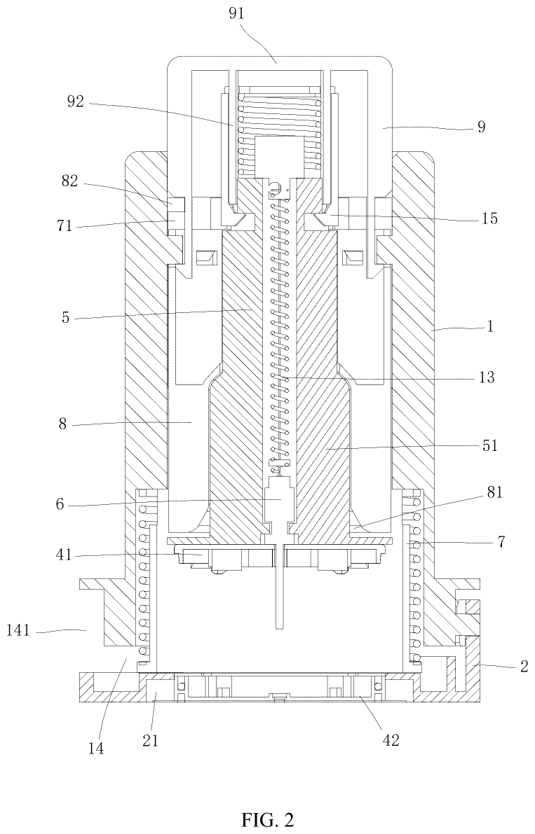

As illustrated in FIG. 1 and FIG. 2, a continuous analyte monitoring device includes an outer housing 1 having a first end and a second end opposite to the first end in a first direction, the second end having an engagement opening 11; a bottom housing 2 connected to the second end and having an implantation opening 21; a monitoring assembly 4 including a first monitoring unit 41 fixed inside the outer housing 1 and a second monitoring unit 42 fixed at the bottom housing 2, the first monitoring unit 41 including a sensor, and the second monitoring unit 42 including a signal processing module; and a sealing assembly 3 configured to abut with the outer housing 1 and/or the bottom housing 2 and located between the first monitoring unit 41 and the second monitoring unit 42 to seal the engagement opening 11. The sealing assembly 3 is configured to move along a second direction to unseal the engagement opening 11 and to allow the engagement opening 11 to communicate with the implantation opening 21. The second direction is perpendicular to the first direction.

In the present disclosure, a driving unit 5 and the first monitoring unit 41 are fixed within the outer housing 1, while the second monitoring unit 42 is fixed within the bottom housing 2. In this way, the continuous analyte monitoring device is divided into two modules. Components having relatively high sterilization requirements, such as a puncture unit 6 and a sensor, are concentrated in one module (within the outer housing 1). Therefore, before leaving the factory, two modules, the outer housing and the bottom housing 2, can be separately sterilized using different processes or at different levels. After the sterilization, the outer housing 1 and the bottom housing 2 are assembled into an entirety, and the sealing assembly 3 is arranged between the first monitoring unit 41 and the second monitoring unit 42 to isolate internal chambers of the outer housing 1 and the bottom housing 2. This prevents an issue of a failure of a sensor or an electronic component during the sterilization. Moreover, since the bottom housing 2 and the outer housing 1 have been fixed in the factory, a user does not need to perform any fixing operations on the bottom housing 2 and the outer housing 1 before use, which reduces operation steps, improving the use experience.

Additionally, in the factory, the bottom housing 2 and the outer housing 1 have already been connected, with a space reserved between the bottom housing 2 and the outer housing 1 for a removal of the sealing assembly 3. In this way, when using the device, the user can realize communication between an inside of the outer housing 1 and an inside of the bottom housing 2 simply by moving the sealing assembly 3 in the second direction, without assembling the bottom housing 2 and the outer housing 1 and without removing sealing films from the bottom housing 2 and the outer housing 1 separately. Therefore, use steps of the product are greatly simplified, which lowers operational difficulty, and makes preparation steps before use simpler and more convenient for the user. Further, before and after a movement of the sealing assembly 3, the outer housing 1 and the bottom housing 2 remain coaxially arranged, with only a gap left between the outer housing 1 and the bottom housing 2. Consequently, when the sealing assembly 3 is laterally removed, the bottom housing 2 still shields the engagement opening 11 of the outer housing 1. Although the engagement opening 11 is in communication with an ambient environment, a path for dust, bacteria, etc., to enter the engagement opening 11 becomes more circuitous due to shielding of the bottom housing 2, which reduces a risk of bacterial entry into the outer housing 1 from the engagement opening 11, enhancing cleanliness of assemblies inside the outer housing 1. Since the sealing assembly 3 moves in the second direction perpendicular to the first direction (an implantation direction), a movement of the sealing assembly 3 does not affect a movement of the driving unit 5 inside the outer housing 1. Therefore, a risk of interference between the sealing assembly 3 and an implantation movement of the driving unit 5 is lowered, which ensures that the driving unit 5 can reliably perform the implantation movement after the sealing assembly 3 unseals the engagement opening 11.

It should be noted that, the first direction extends in an axial direction of the outer housing 1 towards the engagement opening 11, whereas the second direction may extend in a radial direction of the outer housing 1 or any direction not passing through an axis of the outer housing 1, as long as the second direction is perpendicular to the first direction. That is, as illustrated in FIG. 1, when the outer housing 1 is vertically arranged, the first direction extends vertically downwards, and the second direction only needs to extend horizontally and is not limited in this regard. Additionally, the engagement opening 11 can be sealed as long as the sealing assembly 3 is in contact with at least one of the outer housing 1 and the bottom housing 2. That is, the sealing assembly 3 may abut with the second end of the outer housing 1, or with the bottom housing 2, or with the outer housing 1 at a side of the sealing assembly 3 and with the bottom housing 2 at another side of the sealing assembly 3.

It should also be noted that a method for unsealing the engagement opening 11 by the sealing assembly 3 is not limited in the present disclosure. In a preferred embodiment, the sealing assembly 3 is movable in the second direction to be taken out from a space between the outer housing 1 and the bottom housing 2. In this case, the sealing assembly 3 is completely disengaged from an assembly composed of the outer housing 1 and the bottom housing 2. The user can simply discard the sealing assembly 3 taken out. In other embodiments, the sealing assembly 3 may not be completely taken out. For example, the user can simply move the sealing assembly 3 in the second direction by a predetermined distance to disengage the sealing assembly 3 from the outer housing 1 and/or the bottom housing 2 and allow the engagement opening 11 to communicate with the implantation opening 21, without completely taking out the sealing assembly 3. In this case, the sealing assembly 3 remains at the assembly formed by the outer housing 1 and the bottom housing 2. However, the sealing assembly 3 abuts with neither the outer housing 1 nor the bottom housing 2, and avoids the engagement opening 11 and the implantation opening 21 to allow the engagement opening 11 to communicate with the implantation opening 21. In this way, a need for the user to dispose the sealing assembly 3 separately is eliminated, allowing the entire device to be discarded for recycling after an implantation is complete.

As a preferred embodiment of the present disclosure, as illustrated in FIG. 2 and FIG. 7 to FIG. 9, a mounting channel 14 is defined between the outer housing 1 and the bottom housing 2. The mounting channel 14 is configured to at least partially receive the sealing assembly 3. The mounting channel 14 has a mounting opening 141 that extends through the outer housing 1 and/or the bottom housing 2 along the second direction. The mounting opening 141 is configured for the sealing assembly 3 to enter or exit the mounting channel 14. During mounting of the sealing assembly 3 before the device leaves the factory, at least part of the sealing assembly 3 extends through the mounting opening 141 into the mounting channel 14 to abut with the outer housing 1 or the bottom housing 2, for sealing the engagement opening 11. Before use, the user laterally takes out the sealing assembly 3 from the mounting opening 141 to allow the engagement opening 11 to communicate with the implantation opening 21, and thus the continuous analyte monitoring device is in a state waiting for the implantation.

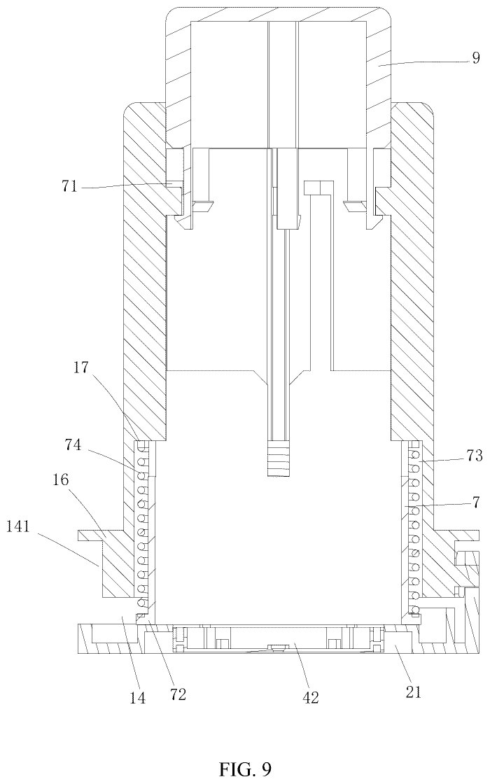

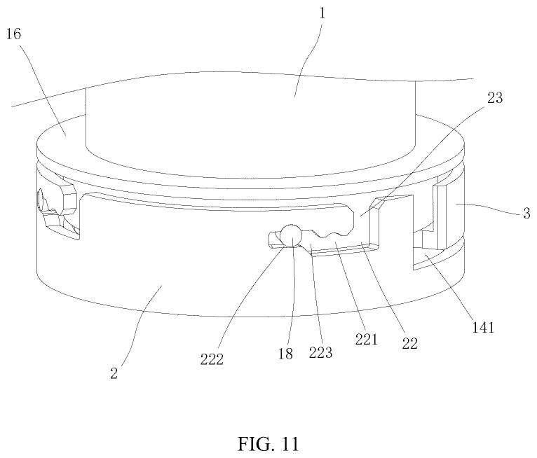

A method for forming the mounting opening 141 is not limited in this embodiment. Preferably, as illustrated in FIG. 2, FIG. 9, and FIG. 11, at least one of the second end of the outer housing 1 and the bottom housing 2 has an avoidance groove. The mounting opening 141 is formed through cooperation with the avoidance groove. That is, the mounting opening 141 is formed through cooperation between the outer housing 1 and the bottom housing 2. Of course, the mounting opening 141 may also be separately formed at the outer housing 1 or the bottom housing 2. Specifically, as illustrated in FIG. 9 and FIG. 11, an outer wall of the outer housing 1 is provided with an engagement rib 16 protruding outwards. The engagement rib 16 is configured to cooperate with the bottom housing 2 to form the mounting opening 141.

It should be noted that the user can take out the sealing assembly 3 through the mounting opening 141. However, a method for mounting the sealing assembly 3 before the device leaves the factory is not limited in the present disclosure. In an embodiment, the sealing assembly 3 is also mounted through the mounting opening 141. Before the device leaves the factory, an assembler may assemble the outer housing 1 and the bottom housing 2, and then insert the sealing assembly 3 into the mounting channel 14 from the mounting opening 141 in a direction opposite to the second direction, in such a manner that the sealing assembly 3 abuts with the outer housing 1 and/or the bottom housing 2 to realize sealing.

In another embodiment, the assembler synchronously mounts the sealing assembly 3 between the outer housing 1 and the bottom housing 2 while assembling the outer housing 1 and the bottom housing 2, and adjusts positions of the outer housing 1 and the bottom housing 2 to clamp the sealing assembly 3 to realize sealing.

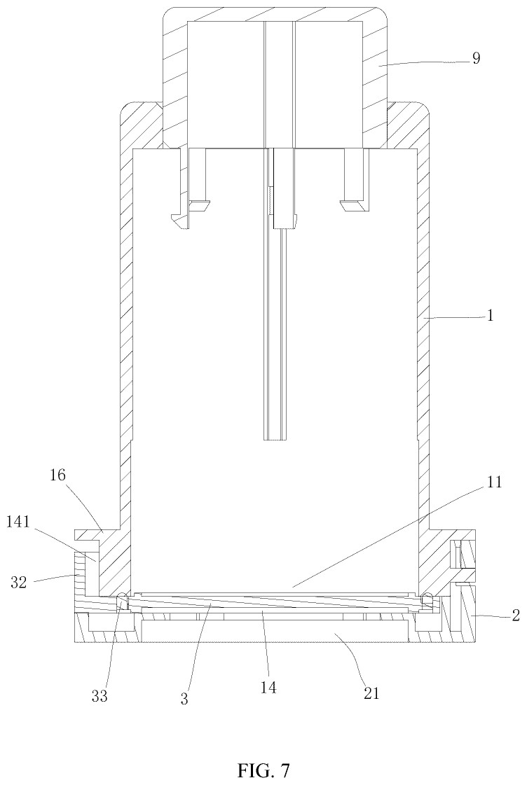

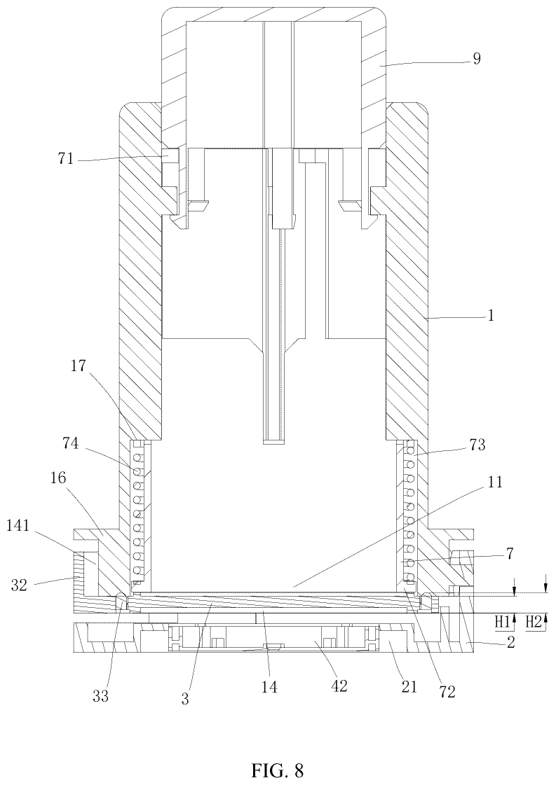

Further, as illustrated in FIG. 1, FIG. 7, and FIG. 8, the sealing assembly 3 includes a sealing portion 31 and a gripping portion 32 extending from the sealing portion 31. The gripping portion 32 protrudes out of the mounting opening 141. The sealing portion 31 is located inside the mounting channel 14 and abuts with the outer housing 1 and/or the bottom housing 2 to seal the engagement opening 11. The gripping portion 32 is located outside the mounting opening 141 and serves as a handle for mounting or removing the sealing assembly 3. The sealing portion 31 can be placed into or taken out of the mounting channel 14 through grasping the gripping portion 32. In this way, grasping and an operation of the sealing assembly 3 are facilitated, which lowers the operational difficulty. In addition, the gripping portion 32 is exposed at an exterior surface of the product, which can also provide an obvious prompt for the user for guiding the user to take out the sealing assembly 3 in a correct direction and with a proper operation, making the operation simpler and clearer and improving the use experience.

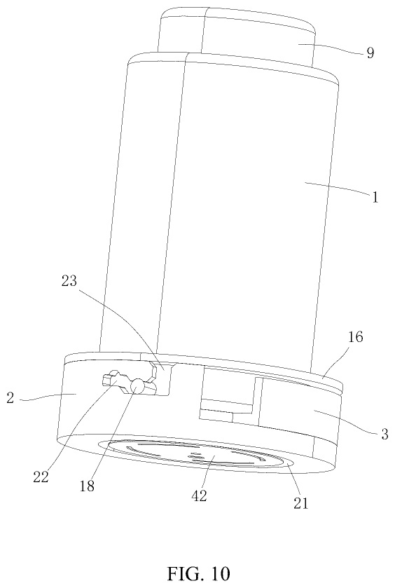

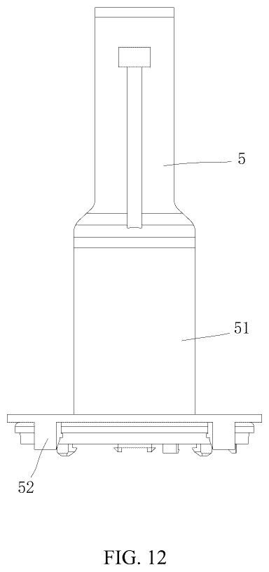

Preferably, as illustrated in FIG. 8, FIG. 10, and FIG. 12, an exterior surface of the gripping portion 32 is flush with the bottom housing 2 and/or the outer housing 1, which on the one hand makes an overall appearance of the continuous analyte monitoring device more uniform and flat and reduces a sense of abruptness, and on the other hand prevents the gripping portion 32 from protruding out and causing the sealing assembly 3 to be shifted in position or the sealing to be ineffective due to a collision during transportation and warehousing.

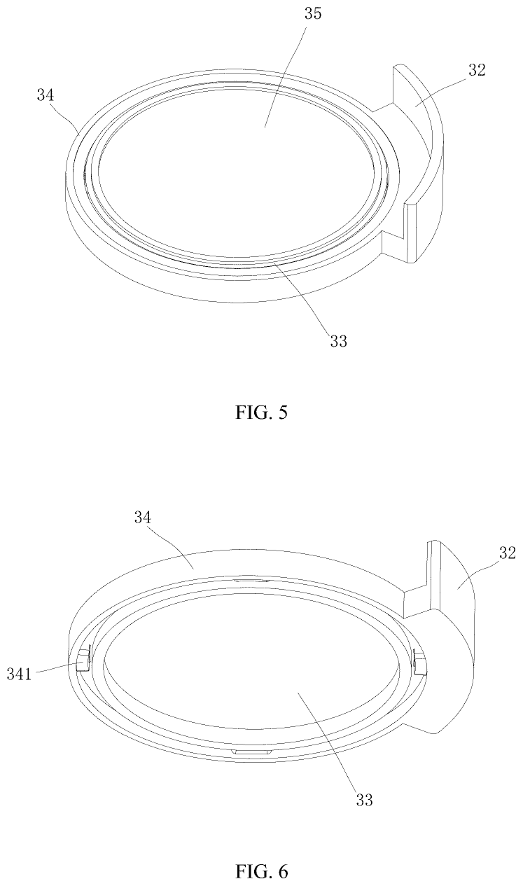

In another preferred embodiment, as illustrated in FIG. 1, FIG. 5, and FIG. 6, the sealing assembly 3 includes a connecting element 34 and a sealing element 33 fixed at the connecting element 34. The sealing element 33 is configured to abut with the outer housing 1 to seal the engagement opening 11. The sealing element 33 is fixed at the connecting element 34. The connecting element 34 serves as a carrier for the sealing element 33. A position of the sealing element 33 can be adjusted by the user through operating the connecting element 34 to enable the sealing element 33 to abut with the outer housing 1 or the bottom housing 2 to realize sealing, or enable the sealing element 33 to disengage from the outer housing 1 or the bottom housing 2. Since the sealing element 33 is mostly made of an elastic material, which is soft and prone to deformation, it is relatively difficult to directly operate the sealing element 33. However, the connecting element 34 can restrict a shape and the position of the sealing element 33, which not only facilitates the operation, but also restricts the position of the sealing element 33 to maintain the sealing element 33 at a predetermined posture, forming stable sealing with the outer housing 1 or the bottom housing 2 to prevent sealing performance from being affected due to an excessive deformation of the sealing element 33. Specifically, as illustrated in FIG. 5 and FIG. 6, the connecting element 34 is provided with a shielding portion 35 configured to shield the engagement opening 11 and has a mounting groove surrounding an outer periphery of the shielding portion 35. The sealing element 33 is disposed in the mounting groove. The mounting groove forms a positional restriction on the sealing element 33 to prevent the position of the sealing element 33 from shifting when the sealing element 33 is pressed. The connecting element 34 is further provided with a snapping hook 341. The sealing element 33 is engaged with the snapping hook 341 to fix the sealing element 33 at the connecting element 34, preventing the sealing element 33 from falling off. Of course, the sealing element 33 may also be fixed at the connecting element 34 by other means, such as gluing, screws, which is not limited in this regard.

As a preferred embodiment of the present disclosure, as illustrated in FIG. 1, FIG. 2, and FIG. 7 to FIG. 9, the outer housing 1 and the bottom housing 2 are configured to be rotatable relative to each other to switch between a first relative position and a second relative position. In the first relative position, the sealing assembly 3 seals the engagement opening 11. In the second relative position, the sealing assembly 3 unseals the engagement opening 11. At the factory, after the assembler mounts the sealing assembly 3 in an appropriate place, the outer housing 1 and the bottom housing 2 are adjusted to form the first relative position, in such a manner that the sealing assembly 3 seals the engagement opening 11. Before use, the bottom housing 2 and the outer housing 1 are switched to form the second relative position by the user through performing a rotation operation to rotate the bottom housing 2 and the outer housing 1 relative to each other. In this case, the sealing assembly 3 unseals the engagement opening 11, and a frictional resistance between the sealing assembly 3 and the outer housing 1 or the bottom housing 2 is therefore reduced, which allows the user to easily remove the sealing assembly 3. In addition, the rotation operation is simpler and more convenient, enhancing ease of use.

Further, in the first relative position, a first gap is formed between the outer housing 1 and the bottom housing 2. In the second relative position, a second gap is formed between the outer housing 1 and the bottom housing 2. The first gap is smaller than the second gap. The bottom housing 2 and the outer housing 1 are configured to be rotatable relative to each other to move towards each other for tightly clamping the sealing assembly 3 or move away from each other for loosening the sealing assembly 3. In the first relative position, the first gap between the outer housing 1 and the bottom housing 2 is relatively small, and thus a clamping force is exerted on the sealing assembly 3. Under such a pressing force, the sealing assembly 3 tightly abuts with the outer housing 1 and/or the bottom housing 2 to form stable sealing. When the outer housing 1 and the bottom housing 2 are rotated relative to each other to form the second relative position, the second gap between the outer housing 1 and the bottom housing 2 is relatively large, which reduces or eliminates the clamping force exerted by the outer housing 1 and the bottom housing 2 on the sealing assembly 3. In this case, the user can easily take out the sealing assembly 3 with a relatively small frictional resistance, making the operation more effortless.

Preferably, as illustrated in FIG. 8, the sealing assembly 3 includes a sealing portion 31 sandwiched between the outer housing 1 and the bottom housing 2. The sealing portion 31 has a thickness H2 greater than or equal to the first gap H1. Since the thickness H2 of the sealing portion 31 is greater than or equal to the first gap H1, the outer housing 1 and the bottom housing 2, when in the first relative position, can press the sealing portion 31 from two sides of the sealing portion 31, ensuring the sealing performance of the sealing portion 31. In addition, a relatively large frictional resistance that restricts the movement of the sealing assembly 3 is generated among the sealing portion 31, the outer housing 1, and the bottom housing 2. In this way, the sealing portion 31 is prevented from being accidentally taken out in advance during transportation or warehousing, preventing cleanliness inside the outer housing 1 from being affected or even leading to a product failure. Only when the user rotates the bottom housing 2 and the outer housing 1 to form the second relative position, the pressing force that the bottom housing 2 and the outer housing 1 exert on the sealing assembly 3 decreases, allowing the sealing portion 31 to move along the second direction.

In a preferred embodiment, as illustrated in FIG. 10 and FIG. 11, the outer housing 1 is provided with a fixing protrusion 18. The bottom housing 2 has a fixing groove 22. The fixing groove 22 has a locking position 221 and an unlocking position 222 spaced apart from the locking position 221 circumferentially. A height difference exists between the locking position 221 and the unlocking position 222 in the first direction. The fixing protrusion 18 is engaged with the fixing groove 22, and is slidable in the fixing groove 22 when the bottom housing 2 and the outer housing 1 rotate relative to each other to provide movement guidance for rotations of the bottom housing 2 and the outer housing 1, standardizing the operation of the user. In this way, the user is only able to rotate the bottom housing 2 and the outer housing 1 relative to each other, preventing the user from operating the bottom housing 2 and the outer housing 1 through pressing or other means. The height difference between the locking position 221 and the unlocking position 222 of the fixing groove 22 allows for converting relative rotations between the bottom housing 2 and the outer housing 1 into axial movements of the bottom housing 2 and the outer housing 1 under guidance of the fixing groove 22. Therefore, the bottom housing 2 and the outer housing 1 can automatically move towards each other to tightly clamp the sealing assembly 3 when the bottom housing 2 and the outer housing 1 are rotated relative to each other to the locking position 221, and the bottom housing 2 and the outer housing 1 can move away from each other to loosen the sealing assembly 3 when the bottom housing 2 and the outer housing 1 are rotated to the unlocking position 222.

In other embodiments, the fixing protrusion 18 may also be disposed at the bottom housing 2. Correspondingly, the fixing groove 22 is formed at the outer housing 1.

Preferably, as illustrated in FIG. 10 and FIG. 11, an end of the bottom housing 2 is arranged around an outer side of the second end of the outer housing 1, and the fixing groove 22 is a through groove that extends through a side wall of the bottom housing 2. In this way, the user can see a position of the fixing protrusion 18 within the fixing groove 22 from the outside, and thus can direct obtain a state of the sealing assembly 3.

Further, as illustrated in FIG. 11, the fixing groove 22 further includes an extension segment 223 arranged between the locking position 221 and the unlocking position 222. The extension segment 223 has a guide surface smoothly connecting the locking position 221 and the unlocking position 222. Smoothly connecting the unlocking position 222 and the locking position 221 by the extension segment 223 enables switching of the fixing protrusion 18 between the locking position 221 and the unlocking position 222 to be smoother, which reduces a frictional resistance between the fixing protrusion 18 and an inner wall of the fixing groove 22 and reduces a sense of jamming generated by a movement of the fixing protrusion 18 relative to the fixing groove 22, making the operation of the user more effortless. Specifically, as illustrated in FIG. 11, the fixing protrusion 18 is arranged in a cylindrical shape. The inner wall of the fixing groove 22 has contact arc surfaces adapted to an outer surface of the fixing protrusion 18 at the unlocking position 222 and the locking position 221. The fixing protrusion 18 in the cylindrical shape can reduce a friction between the fixing protrusion 18 and a groove wall of the fixing groove 22 to realize a smoother movement of the fixing protrusion 18 in the fixing groove 22. In this way, the operation of the user is more effortless, and the sense of jamming is reduced. Since each of the unlocking position 222 and the locking position 221 has the contact arc surface, a better engagement with the fixing protrusion 18 can be realized to restrict the fixing protrusion 18 to the unlocking position 222 or the locking position 221.

In other embodiments, the fixing protrusion 18 may also be of other shapes. A shape of the contact surface is adjusted based on a shape of the outer surface of the fixing protrusion 18, in such a manner that shapes of the fixing protrusion 18 and the fixing groove 22 are adapted to each other to form an engagement. The contact arc surface may be formed at a groove wall of the fixing groove 22 at a side of the fixing groove 22, or the contact arc surfaces may be formed at groove walls of the fixing groove 22 at two sides of the fixing groove 22. The present disclosure is not limited in this regard.

Further, the bottom housing 2 has a guiding channel 23 in communication with the fixing groove 22 and at least partially extending through the bottom housing 2 along the first direction. The fixing protrusion 18 is configured to enter the fixing groove 22 from the guiding channel 23. The guiding channel 23 is configured to allow the fixing protrusion 18 to enter or slide out of the fixing groove 22. During assembly of the outer housing 1 and the bottom housing 2, the fixing protrusion 18 is made to slide from the guiding channel 23 into the fixing groove 22 in the first direction, and then the bottom housing 2 and the outer housing 1 are rotated relative to each other to rotate the fixing protrusion 18 to the locking position 221.

In a preferred embodiment, as illustrated in FIG. 1 and FIG. 8, the first end is equipped with a trigger unit 9, and the outer housing 1 is further equipped with a locking member 7 inside the outer housing 1. The locking member 7 is configured to abut between the trigger unit 9 and the sealing assembly 3 to prevent the trigger unit 9 from moving towards the sealing assembly 3. The locking member 7 is capable of forming a movement restriction on the trigger unit 9 to prevent the trigger unit from moving, and thus the continuous analyte monitoring device cannot be triggered, which serves to prevent accidental triggering before the product is used. In addition, the locking member 7 is configured to abut with the sealing assembly 3, in such a manner that the sealing assembly 3 forms a movement restriction on the locking member 7. That is, when the sealing assembly 3 is in a sealing state, the locking member 7 abuts with the trigger unit 9 to prevent the device from being triggered. Only when the user operates the sealing assembly 3 to move to the position in which the sealing assembly 3 unseals the engagement opening 11, the sealing assembly 3 loses the restriction on the locking member 7. Therefore, the locking member 7 moves and therefore loses the restriction on the trigger unit 9, in this case, the trigger unit 9 can be triggered. In this way, associating the sealing assembly 3 with a state of the locking member 7 ensures that the device can be triggered only after the user takes out the sealing assembly 3, which greatly reduces a probability that the device is scrapped before use due to accidental triggering caused by a collision or pressing, improving reliability of the product. In this way, the sealing assembly 3 of the present disclosure can not only provide the sealing, but also cooperate with the locking member 7 to prevent the accidental triggering of the trigger unit 9. Such a multi-purpose design simplifies an internal structure of the device and reduces costs.

Specifically, as illustrated in FIG. 1, FIG. 2, FIG. 8, and FIG. 9, the locking member 7 extends in the first direction to form an acting end and a stop end. The acting end is configured to abut with the sealing assembly 3. The stop end is configured to lock the trigger unit 9. The locking member 7 extends in the first direction, in such a manner that an end of the locking member 7 is located at the first end and abuts with the trigger unit 9, and another end of the locking member 7 is located at the second end and abuts with the sealing assembly 3. Under an abutment force exerted by the sealing assembly 3 on the acting end, the stop end firmly abuts with the trigger unit 9 to prevent the trigger unit 9 from moving. When the sealing assembly 3 is taken out, the acting end loses an abutment with the sealing assembly 3, allowing the locking member 7 to move as a whole in the first direction. Consequently, the stop end is disengaged from the trigger unit 9, and the trigger unit 9 is therefore free from a restriction and can be triggered to move along the first direction.

Specifically, as illustrated in FIG. 1 and FIG. 2, the outer housing 1 is further provided with a support member inside the outer housing 1. The support member and at least part of the trigger unit 9 are engaged with each other to form a sliding groove 82 extending in the first direction. The stop end includes a stop protrusion 71 located in the sliding groove 82. In a locking position, the stop protrusion 71 abuts with the trigger unit 9. When the stop protrusion 71 moves to the release position in the first direction, the stop protrusion 71 is disengaged from the trigger unit 9 and rests against the support member, forming a movement restriction on the locking member 7 in the first direction to prevent the locking member 7 from slipping off. Therefore, a length of the sliding groove 82 in the first direction is a movement stroke of the locking member 7.

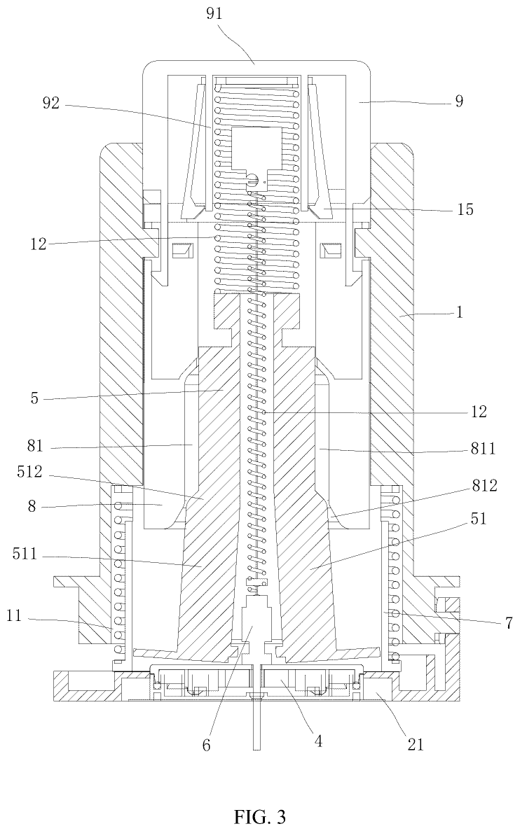

Preferably, as illustrated in FIG. 1, the outer housing 1 is provided with a limiting sleeve 8 inside the outer housing 1, and the driving unit 5 is located in a limiting channel 81 within the limiting sleeve 8. The limiting sleeve 8 constitutes the support member. Specifically, as illustrated in FIG. 2 and FIG. 3, the outer housing 1 is internally provided with the driving unit 5, the driving unit 5 is configured to carry the puncture unit 6 to move along the first direction to perform an implantation operation. The trigger unit 9 includes a trigger button 91 and a trigger rib 92. The trigger button 91 is fixed to the outer housing 1. The outer housing 1 is internally provided with an elastic claw 15 in a stop engagement with the driving unit 5. The elastic claw 15 surrounds an outer side of the trigger rib 92. When the user presses the trigger button 91 in the first direction, the trigger rib 92 presses the elastic claw 15, in such a manner that the elastic claw 15 expands and deforms outwards or moves and is therefore disengaged from the driving unit 5, completing a release of the driving unit 5.

Further, a first receiving channel is formed by the trigger rib 92, and the driving unit 5 has a second receiving channel inside the driving unit 5. The first receiving channel is internally provided with a propelling member 12 configured to drive the driving unit 5 to move along the first direction. The second receiving channel is internally provided with a restoring member 13 configured to drive the puncture unit 6 to move along a third direction, the third direction being opposite to the first direction. The restoring member 13 and the propelling member 12 are coaxially arranged. In this way, movements and deformations of the propelling member 12 and the restoring member 13 are restricted to the two channels to improve movement guidance for the propelling member 12 and the restoring member 13, allowing the puncture unit 6 to move smoothly and reducing shaking.

Preferably, each of the propelling member 12 and the restoring member 13 is a spring. It should be noted that a method for driving the locking member 7 to move along the first direction after the sealing assembly 3 is taken out is not limited in the present disclosure. In an embodiment, when the continuous analyte monitoring device is in use, the outer housing 1 is vertically arranged and the implantation opening 21 abuts with the skin surface of the user. When the sealing assembly 3 is taken out, the locking member 7 loses upward support and therefore moves downwards in the first direction under gravity of the locking member 7, unlocking the trigger unit 9.

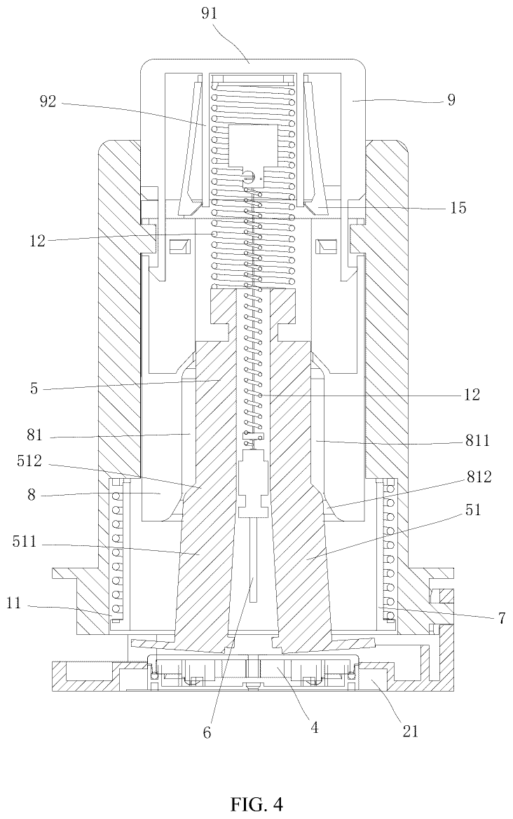

In another embodiment, as illustrated in FIG. 1 to FIG. 4 and FIG. 8 to FIG. 9, the outer housing 1 is further equipped with a pushing member 74 inside the outer housing 1. The pushing member 74 is configured to apply a thrust in the first direction to the locking member 7. When the pushing member 74 applies the thrust in the first direction to the locking member 7 to enable the locking member 7 to abut with the sealing assembly 3, the pushing member 74 is in an energy storage state. When the sealing assembly 3 no longer abuts with the locking member 7, the pushing member 74 is released and pushes the locking member 7 to move quickly in the first direction, removing the movement restriction on the trigger unit 9. In this way, the locking member 7 is able to react quickly to unlock the trigger unit 9 after the sealing assembly 3 is taken out. Therefore, response sensitivity of the locking member 7 is improved to reduce waiting time for the user, which in turn shortens a duration of the entire implantation process, making the implantation process smoother and improving the use experience.

Further, the pushing member 74 is arranged between an inner wall of the outer housing 1 and an outer wall of the locking member 7. Specifically, as illustrated in FIG. 8 and FIG. 9, the outer housing 1 has a receiving groove 17 at an inner wall of the second end. The locking member 7 is provided with an outer flanging 72 at an end of the locking member 7 facing towards the implantation opening 21 to form a receiving space 73 for receiving the pushing member 74. The pushing member 74 has an end abutting with a top wall of the receiving groove 17 and another end abutting with the outer flanging 72. Preferably, the pushing member 74 is a spring to save costs.

As a preferred embodiment of the present disclosure, as illustrated in FIG. 1 to FIG. 4, the continuous analyte monitoring device further includes the driving unit 5 and the puncture unit 6 that are disposed inside the outer housing 1. The trigger unit 9 is disposed at the first end. Activating the trigger unit 9 causes the driving unit 5 to drive the puncture unit 6 to move along the first direction, to cause the first monitoring unit 41 to electrically connect the second monitoring unit 42 and cause the sensor to be partially inserted into a host.

After activating the trigger unit by the user, the first monitoring unit 41 can move with the driving unit 5 and the puncture unit 6 in the first direction. The first monitoring unit 41 and the second monitoring unit 42 synchronously complete an electrical connection while the puncture unit 6 carries the sensor inside the first monitoring unit 41 to be inserted into the body of the host. In this way, assembly of the monitoring assembly 4, the electrical connection between the first monitoring unit 41 and the second monitoring unit 42, and an implantation of the sensor are simultaneously accomplished by activating the trigger unit. Therefore, operation steps of the user in the implantation process are simplified, the operational difficulty of the product is lowered, and learning costs of the user are lowered. Further, a duration of the entire implantation stage is shortened, which helps to reduce fear of the user while waiting for the implantation. When the continuous analyte monitoring device of the present disclosure is in use, the user only needs to activate the trigger unit to simultaneously realize both the electrical connection between the first monitoring unit 41 and the second monitoring unit 42 and the implantation of the sensor. In this way, the whole implantation process is completed, improving the use experience.

It should be noted that a time sequence in which the first monitoring unit 41 and the second monitoring unit 42 are electrically connected and the sensor is partially inserted into the body of the host is not limited in the present disclosure. For example, during movements of the driving unit 5, the puncture unit 6, and the first monitoring unit 41 together in the first direction, the first monitoring unit 41 and the second monitoring unit 42 may be electrically connected first, at which time the driving unit 5 and the puncture unit 6 have not yet been moved into an appropriate place. As the movements continue, the puncture unit 6 is inserted into the body of the host to complete the implantation. That is, the electrical connection between the first monitoring unit 41 and the second monitoring unit 42 occurs before the puncture unit 6 is inserted into the body of the host. For example, during the movements of the driving unit 5, the puncture unit 6, and the first monitoring unit 41 together in the first direction, the puncture unit 64 is inserted into the body of the host first, at which time the driving unit 5 and the puncture unit 6 have not yet been moved into an appropriate place. As the movements continue, the first monitoring unit 41 and the second monitoring unit 42 complete the electrical connection. That is, the insertion of the puncture unit 6 into the body of the host occurs before the electrical connection between the first monitoring unit 41 and the second monitoring unit 42 occurs. In addition, the above two steps may also occur simultaneously.

However, regardless of the situations in any of the above examples, the user only needs to activate the trigger unit, without carrying out any additional operations. Consequently, all the situations described in the above examples fall within the scope of protection of the present disclosure.

Preferably, the continuous analyte monitoring device of the present disclosure further includes a bottom cover detachably connected to the bottom housing 2. The bottom cover is configured to cover the implantation opening 21. Before leaving the factory, a sealed cavity is formed by the bottom cover, the outer housing 1, and the bottom housing 2 together. Before use, the user removes the bottom cover from the bottom housing 2 to expose the implantation opening 21, and then places the implantation opening 21 against the skin surface, allowing the implantation operation to be performed.

It should be noted that the present disclosure does not impose any limitations on a driving structure and a positional restriction structure of the driving unit 5 for the puncture unit 6. In a preferred embodiment, as illustrated in FIG. 1 to FIG. 4, the continuous analyte monitoring device further includes the driving unit 5 and the puncture unit 6 that are disposed inside the outer housing 1. The driving unit 5 includes at least two clamping portions 51. Each of the at least two clamping portions 51 is located at an outer side of the puncture unit 6 to restrict a movement of the puncture unit 6 relative to the clamping portion 51. The outer housing 1 is further provided with the limiting sleeve 8 inside the outer housing 1. The limiting sleeve 8 has the limiting channel 81 extending in the first direction. The clamping portion 51 is located in the limiting channel 81 and movable within the limiting channel 81 in the first direction. When the clamping portion 51 moves relative to the limiting channel 81 to a release position, the clamping portion 51 releases the puncture unit 6 to enable the puncture unit 6 to move relative to the clamping portion 51 in a third direction, the third direction being opposite to the first direction.

The clamping portion 51 realizes a restriction on the puncture unit 6 in the limiting channel 81 by means of clamping and fixation. The clamping portion 51 is configured to tightly clamp the puncture unit 6 under an abutment action of an inner wall of the limiting channel 81 at the outer side, in such a manner that the puncture unit 6 maintains a constant position relative to the driving unit 5. With the clamping force, on the one hand, the trigger unit 9 can drive the puncture unit 6 when the driving unit 5 is triggered, for carrying the puncture unit 6 to move together in the first direction. On the other hand, the puncture unit 6 can be firmly fixed to restrict a position of the puncture unit 6, which prevents the puncture unit 6 from performing a needle withdrawal action due to being released in advance, ensuring that the entire implantation process is carried out reliably and orderly. With a movement of the clamping portion 51 in the first direction, at least part of the clamping portion 51 slides out of the limiting channel 81. Therefore, pressing from the inner wall of the limiting channel 81 disappears, and the clamping force of the clamping portion 51 on the puncture unit 6 is weakened. Consequently, the puncture unit 6 is free from the restriction of the driving unit 5 and is released, and then moves in the third direction to completes the needle withdrawal.

Driving the puncture unit 6 and restricting or triggering a needle reaction movement in a manner of tightly clamping or releasing the puncture unit 6 by the clamping portion 51 from the outer side simplifies structural designs of the driving unit 5 and the puncture unit 6 and eliminates a need to dispose complicated stop structures at the driving unit 5 and the puncture unit 6. In addition, positional requirements for each component are lowered, and requirements for manufacturing accuracy and assembly accuracy are reduced, which reduces production difficulty and improves a production efficiency. Further, along a path where the puncture unit 6 moves in the first direction for a needle insertion and in the third direction for a needle withdrawal, there are no structures oriented perpendicularly to the first direction that would interfere with the movement of the puncture unit 6, which in turn improves stability and smoothness during the movement of the puncture unit 6, enabling the puncture unit 6 to move smoothly and switching between the needle insertion and the needle withdrawal to be performed more smoothly without generating any sense of jamming. Furthermore, shaking generated during the movement of the puncture unit 6 is also reduced, which enables the puncture unit 6 to moves more smoothly in the axial direction of the outer housing 1, reducing a sensation of pain for the user during use.

Preferably, two clamping portions 51 are provided and arranged opposite to each other at two sides of the puncture unit 6. The two clamping portions 51 are configured to jointly move inwards to clamp the puncture unit 6 and jointly move outwards to release the puncture unit 6. Of course, also, more than two clamping portions 51 may be provided and evenly arranged at intervals in a circumferential direction of the puncture unit 6 to improve deformation performance of the clamping portions 51, exerting a stable and uniform clamping force on the puncture unit 6. The limiting sleeve 8 is arranged around an outer side of the clamping portion 51.

It should be noted that a shape of a cross section of the limiting sleeve 8 is not limited in the present disclosure. Preferably, the limiting sleeve 8 is of a cylindrical shape and has a circular cross section. The cross section of the limiting sleeve 8 may be also of other shapes, e.g., a rectangular shape, a triangular shape, a polygonal shape, and so on. The present disclosure is not limited in this regard.

Preferably, as illustrated in FIG. 3, the limiting sleeve 8 includes a clamping segment 811 and a release segment 812. In the first direction, a cross-sectional area of the clamping segment 811 remains constant and a cross-sectional area of the release segment 812 gradually increases. The clamping portion 51 includes an inclined segment 512 and a fixed segment 511. In the first direction, a cross-sectional area of the fixed segment 511 remains constant and a cross-sectional area of the inclined segment 512 gradually increases.

Specifically, as illustrated in FIG. 3, the release segment 812 is disposed at an end of the clamping segment 811 facing towards the second end, and the inclined segment 512 is disposed at an end of the fixed segment 511 facing towards the first end.

As illustrated in FIG. 2 and FIG. 3, both the clamping portion 51 and the puncture unit 6 are disposed within the clamping segment 811 of the limiting sleeve 8 in respective initial positions of the clamping portion 51 and the puncture unit 6. The fixed segment 511 of the clamping portion 51 is pressed inwards by the clamping segment 811 to tightly clamp the puncture unit 6. The clamping segment 811 and the fixed segment 511 have a same cross-sectional area in the first direction, and thus a uniform pressing force is exerted on the clamping portion 51, which enables the clamping portion 51 to clamp the puncture unit 6 more stably. As the driving unit 5 carries the puncture unit 6 to move along the first direction to the release segment 812, as illustrated in FIG. 3, a position of the inclined segment 512 corresponds to that of the release segment 812. As illustrated in FIG. 3, the cross-sectional area of the release segment 812 gradually increases to form a guiding surface at a side facing towards the driving unit 5. Similarly, the cross-sectional area of the inclined segment 512 of the clamping portion 51 also gradually increases in the first direction to form an engagement surface. The guiding surface is engaged with the engagement surface to guide the clamping portion 51 to move outwards for releasing the puncture unit 6. A change in the cross-sectional area of the release segment 812 in the first direction can guide the clamping portion 51 to move outwards. Therefore, by disposing the release segment 812 at an end of the limiting channel 81, a release action for the puncture unit 6 is only performed when the at least part of the clamping portion 51 moves in the first direction to the end of the limiting channel 81. Prior to this, the clamping portion 51 firmly clamps the puncture unit 6. In this way, the needle withdrawal action is ensured to be triggered only when the puncture unit 6 completes a needle insertion action, which prevents the needle withdrawal action from being triggered in advance due to the puncture unit 6 being released in advance, ensuring that the entire implantation process is carried out orderly, and improving the reliability of the product.

Preferably, the clamping portion 51 may be made of plastic or other materials having a predetermined elastic deformation capability, which can not only tightly clamp the puncture unit 6 when pressed by the inner wall of the limiting sleeve 8, but also move outwards to release the puncture unit 6 when guided by the release segment 812. The clamping portion 51 may also be made of a material having greater elasticity, such as silicone, to allow for a more rapid deformation and a greater deformation amplitude, triggering the needle withdrawal action timely.

It should be noted that the deformation referred to herein means that the entire clamping portion 51 undergoes the deformation, causing the at least part of the clamping portion 51 to move away from the puncture unit 6. That is, clamping or releasing of the puncture unit 6 by the clamping portion 51 is realized through the movement, and the deformation is intended to move the clamping portion 51. In other embodiments, the driving unit 5 may also be provided with a stop portion configured to abut with an end of the puncture unit 6 facing towards the first end to push the puncture unit 6 to move. In addition, an unlocking structure is disposed at a movement path of the stop portion. In this way, after moving to the release position, the driving unit 5 moves or deforms due to an abutment with the unlocking structure and is disengaged from the puncture unit 6 for releasing the puncture unit 6. The present disclosure is not limited in this regard.

It should be noted that a position for fixing the first monitoring unit 41 is not limited in the present disclosure. In a preferred embodiment, as illustrated in FIG. 1 and FIG. 12, the first monitoring unit 41 is fixed at the driving unit 5. Further, the first monitoring unit 41 is in interference fit with the driving unit 5. Specifically, the driving unit 5 is provided with a plurality of elastic ribs 52 configured to clamp the first monitoring unit 41 together. The elastic rib 52 is configured to release the first monitoring unit 41 when the clamping portion 51 moves outwards. Of course, the first monitoring unit 41 may also be fixed at the driving unit 5 by other means such as a snap connection. Alternatively, a fixing structure may also be disposed at other components or the outer housing 1 to fix the first monitoring unit 41, as long as the first monitoring unit 41 can move along with the driving unit 5 in the first direction.

In addition, a method for fixing the second monitoring unit 42 with the bottom housing 2 is also not limited in the present disclosure, which can also be realized by means of interference fit, a snap connection, and the like.

Preferably, the first monitoring unit 41 and the second monitoring unit 42 may be fixed in the first direction, and are to be assembled into the complete monitoring assembly 4 in the implantation process.

As illustrated in FIG. 1 to FIG. 4, the second monitoring unit 42 is disposed at the implantation opening 21. An adhesive layer of the second monitoring unit 42 is flush with the implantation opening 21. When the user enables the implantation opening 21 to abut with the skin surface, the adhesive layer adheres to the skin. As the first monitoring unit 41 moves in the first direction to complete assembly with the second monitoring unit 42, the second monitoring unit 42 is pressed towards the skin under a pushing force of the first monitoring unit 41 in the first direction to adhere the adhesive layer to the skin. As the outer housing 1 and the bottom housing 2 are removed, the second monitoring unit 42 is disengaged from the bottom housing 2. Of course, the second monitoring unit 42 may also be disposed at a position away from the implantation opening 21 by a predetermined distance. When the first monitoring unit 41 moves to the second monitoring unit 42, the first monitoring unit 41 and the second monitoring unit 42 are assembled and electrically connected. Then, the second monitoring unit 42 is disengaged from the bottom housing 2 under push of the first monitoring unit 41. The monitoring assembly 4 as a whole continues to move along the first direction, until the adhesive layer of the second monitoring unit 42 is fixed to the skin of the host.

In a preferred embodiment, the first monitoring unit 41 further includes a battery configured to be electrically connected to the second monitoring unit 42. The battery mainly serves to supply power to the signal processing module. Before the user triggers the implantation, the battery is not electrically connected to the signal processing module since the first monitoring unit 41 and the second monitoring unit 42 are separated from each other. The battery is electrically connected to the signal processing module only after the user triggers the implantation. In this way, an energy loss of the battery before the device is used by the user can be reduced to extend a warehousing duration.

While some embodiments of the present disclosure have been described above, the present disclosure is not limited to these embodiments. Any modifications, equivalent replacements, and improvements made within the spirit and the principle of the present disclosure shall fall within the scope of the claims of the present disclosure.

What is claimed is:

1. A continuous analyte monitoring device, comprising:
an outer housing having a first end and a second end opposite to the first end in a first direction, the second end having an engagement opening;
a bottom housing connected to the second end and having an implantation opening;
a monitoring assembly comprising a first monitoring unit fixed inside the outer housing and a second monitoring unit fixed at the bottom housing, the first monitoring unit comprising a sensor, and the second monitoring unit comprising a signal processing module; and
a sealing assembly configured to abut with the outer housing and/or the bottom housing and located between the first monitoring unit and the second monitoring unit to seal the engagement opening, the sealing assembly being configured to move along a second direction to unseal the engagement opening and to allow the engagement opening to communicate with the implantation opening, the second direction being perpendicular to the first direction.

2. The continuous analyte monitoring device according to claim 1, wherein a mounting channel is defined between the outer housing and the bottom housing, the mounting channel being configured to at least partially receive the sealing assembly and having a mounting opening that extends through the outer housing and/or the bottom housing along the second direction.

3. The continuous analyte monitoring device according to claim 2, wherein the sealing assembly comprises a sealing portion and a gripping portion extending from the sealing portion and protruding out of the mounting opening.

4. The continuous analyte monitoring device according to claim 1, wherein the sealing assembly comprises a connecting element and a sealing element fixed at the connecting element, the connecting element being configured to support and fix the sealing element, the sealing element being configured to abut with the outer housing to seal the engagement opening.

5. The continuous analyte monitoring device according to claim 1, wherein the outer housing and the bottom housing are configured to be rotatable relative to each other to switch between a first relative position in which the sealing assembly seals the engagement opening and a second relative position in which the sealing assembly unseals the engagement opening.

6. The continuous analyte monitoring device according to claim 1, wherein the outer housing and the bottom housing are configured to be rotatable relative to each other to switch between a first relative position and a second relative position, in the first relative position, a first gap is formed between the outer housing and the bottom housing, and in the second relative position, a second gap is formed between the outer housing and the bottom housing, the first gap being smaller than the second gap.

7. The continuous analyte monitoring device according to claim 6, wherein the sealing assembly comprises a sealing portion sandwiched between the outer housing and the bottom housing and having a thickness greater than or equal to the first gap.

8. The continuous analyte monitoring device according to claim 1, wherein the outer housing is provided with a fixing protrusion, and the bottom housing has a fixing groove having a locking position and an unlocking position spaced apart from the locking position circumferentially, a height difference existing between the locking position and the unlocking position in the first direction.

9. The continuous analyte monitoring device according to claim 8, wherein the fixing groove further comprises an extension segment arranged between the locking position and the unlocking position and having a guiding surface smoothly connecting the locking position and the unlocking position.

10. The continuous analyte monitoring device according to claim 1, wherein the first end is equipped with a trigger unit, and the outer housing is equipped with a locking member inside the outer housing, the locking member being configured to abut between the trigger unit and the sealing assembly to prevent the trigger unit from moving towards the sealing assembly.

11. The continuous analyte monitoring device according to claim 10, wherein the locking member extends in the first direction to form an acting end configured to abut with the sealing assembly and a stop end configured to lock the trigger unit.

12. The continuous analyte monitoring device according to claim 10, wherein the outer housing is further equipped with a pushing member configured to apply a thrust in the first direction to the locking member.

13. The continuous analyte monitoring device according to claim 1, further comprising a driving unit and a puncture unit that are disposed inside the outer housing, wherein the first end is equipped with a trigger unit, activating the trigger unit causes the driving unit to drive the puncture unit to move along the first direction, to cause the first monitoring unit to electrically connect the second monitoring unit and cause the sensor to be partially inserted into a host.

14. The continuous analyte monitoring device according to claim 1, further comprising a driving unit, a puncture unit and a limiting sleeve that are disposed inside the outer housing, the driving unit comprising at least two clamping portions, the limiting sleeve having a limiting channel extending in the first direction, the clamping portions being located in the limiting channel to restrict a movement of the puncture unit relative to the clamping portion.

15. The continuous analyte monitoring device according to claim 14, wherein the limiting sleeve comprises a clamping segment and a release segment, wherein in the first direction, a cross-sectional area of the clamping segment remains constant and a cross-sectional area of the release segment gradually increases, and the clamping portions comprise an inclined segment and a fixed segment, wherein in the first direction, a cross-sectional area of the fixed segment remains constant and a cross-sectional area of the inclined segment gradually increases.

16. A continuous analyte monitoring device, comprising:
an outer housing having a first end and a second end opposite to the first end in a first direction, the second end having an engagement opening;
a monitoring assembly comprising a first monitoring unit fixed inside the outer housing and a second monitoring unit spaced apart from the first monitoring unit, the first monitoring unit comprising a sensor, and the second monitoring unit comprising a signal processing module; and
a sealing assembly located between the first monitoring unit and the second monitoring unit to seal the engagement opening.

17. The continuous analyte monitoring device according to claim 16, wherein the first end is equipped with a trigger unit, and the outer housing is equipped with a locking member inside the outer housing, the locking member being configured to abut between the trigger unit and the sealing assembly to prevent the trigger unit from moving towards the sealing assembly.

18. The continuous analyte monitoring device according to claim 16, wherein the sealing assembly comprises a connecting element and a sealing element fixed at the connecting element, the connecting element being configured to support and fix the sealing element, the sealing element being configured to abut with the outer housing to seal the engagement opening.

19. The continuous analyte monitoring device according to claim 16, wherein the sealing assembly is configured to move along a second direction to unseal the engagement opening, and the second direction being perpendicular to the first direction.

20. A continuous analyte monitoring device, comprising:
an outer housing having a first end and a second end that are arranged opposite to the first end in a first direction, the second end having an engagement opening;
a bottom housing connected to the second end and having an implantation opening;
a monitoring assembly comprising a first monitoring unit fixed inside the outer housing and a second monitoring unit fixed at the bottom housing, the first monitoring unit comprising a sensor, and the second monitoring unit comprising a signal processing module; and
a sealing assembly located between the first monitoring unit and the second monitoring unit to seal the engagement opening, the sealing assembly being configured to move to allow the engagement opening to communicate with the implantation opening.
